(19) United States
(12) Patent Application Publication
(10) Pub. No.: US 2009/0075305 A1
(43) Pub. Date: Mar. 19, 2009

(54) Diagnostic serum antibody profiling

(75) Inventors: Brian Liu, Somerville, MA (US); Shuzhen Qin, West Roxbury, MA (US); Joshua Ehrlich, Duluth, GA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Womern's Hospital, Inc., Boston, MA (US)

(21) Appl. No.: 11/919,703

(22) PCT Filed: May 1, 2006
(86) PCT No.: PCT/US2006/016543

§ 371 Date: Feb. 13, 2008

(51) Int. Cl. G01N 33/53

(52) U.S. Cl. 435/7.92; 435/7.1

(57) ABSTRACT

The invention is directed to a microarray assay procedure that can be used for profiling the antibodies present in serum, plasma or blood. The assay may be used to identify antibodies and antigens that are characteristic of particular diseases or conditions. In addition, the invention includes specific antigens that are associated with prostate cancer, progressive benign prostate hyperplasia (BPH) and ovarian cancer.

### FIELD OF THE INVENTION

The present invention is directed to assays that can be used to compare the serum antibody profile of subjects. The assays may be used to identify patients with cancer and to predict the progress of benign prostate hyperplasia (BPH). In addition, the invention is directed to biomarkers that have been identified using the assays that are associated with prostate cancer, ovarian cancer and progressive BPH.

### BACKGROUND OF THE INVENTION

Prostate cancer is one of the most common malignancies in the United States and, after lung cancer, is the leading cause of cancer-related deaths in men. Currently, the most widely used diagnostic assay for prostate cancer involves measuring the amount of prostate-specific antigen (PSA) in a serum sample. However, this test fails to detect cancer in many men with early stage disease. In addition, there are other prostate-related conditions that can lead to elevated PSA levels and, as a result, only 25-30% of men biopsied for prostate cancer due to an elevated PSA test result are actually found to have the disease.

One particularly difficult problem is in distinguishing between men that have benign prostatic hyperplasia (BPH) and cancer. These conditions often produce similar symptoms, including elevated serum PSA levels (Brower, C A Cancer J. Clin. 49:264-281 (1999)). In addition, there is an emerging body of knowledge indicating that BPH itself is a progressive condition in many men. Progression of BPH may adversely affect the quality of life and interfere with activities of daily living. Although less common, progression of BPH may also lead to acute urinary retention, a need for surgery, urinary incontinence, recurrent urinary tract infections, or obstructive uropathy (Fong, et al., Curr. Opin. Urol. 15:35-38, (2005)). A recent Medical Therapy of Prostatic Symptoms (MTOPS) study demonstrated that therapy with the alpha-adrenergic-receptor antagonist doxazosin or the 5α-reductase inhibitor finasteride, alone or in combination, can delay or prevent clinical progression of BPH (McConnell, et al, N. Engl. J. Med. 349:2387-2398 (2003)). Thus, identifying men with progressive disease could have clinical utility in directing therapy to those most likely to benefit from a preventive approach.

Diagnosing and predicting the outcome of diseases such as prostate cancer and BPH based on serum profiling is a particularly attractive concept. Almost all cells in the body communicate with the blood directly or through extracellular fluids, and many release at least part of their contents into the blood stream upon damage or death. It is difficult to argue convincingly that there is any disease state that does not produce some specific pattern of protein change in the serum. However, the widely divergent amounts of protein present in serum presents a serious technical challenge with respect to its use diagnostically. A few proteins, in particular serum albumin and immunoglobulins, are so dominant that they mask detection of all other proteins (Anderson, et al., Mol. Cell. Proteomics 1(11):845-867 (2002)). As a result, current technologies are unable to analyze many lower-abundance proteins that may serve as biomarkers.

One alternative that may improve sensitivity is to focus on serum antibodies. Proteins not present in normal cells may elicit a host immune response, which affords a dramatic amplification of signal in the form of antibodies relative to the amount of the corresponding antigen. Recently, Zheng et al. demonstrated the presence of serum autoantibodies to a panel of known antigens in various human cancers, including prostate cancer (Zhang, et al., Cancer Epidemiol. Biomarkers Prev. 12:136-143 (2003); Zhang, Cancer Detect. Prev. 28(2):114-118 (2004)). The results strongly suggest that uniquely constituted antigen panels or protein microarrays might provide an approach for discriminating autoantibody reactivity between cancer patients and control individuals.

However, microarray procedures aimed at antibody profiling have their own drawbacks. Many use antigens that have either been chemically synthesized or attached directly to supports. As such, posttranslational and conformational antigenic determinants are lost. It has also been unclear to what extent serum profiling may be successfully used for non-malignant conditions such as BPH.

### SUMMARY OF THE INVENTION

General Summary

The present invention is based upon the development of a microarray assay for examining the antibody profile of a sample of blood, plasma or serum. The main characteristics of the assay are that a group of standard antibodies of known specificity (preferably monoclonal antibodies which recognize a single antigen) are bound to a support, such as a glass slide, with each antibody at a separate location. The corresponding antigens are then bound to the immobilized monoclonal antibodies, e.g., by incubating a crude cell lysate with the prepared support. In this way, a microarray is formed in which antigens maintaining their native structural characteristics are immobilized, each antigen at a unique site on the assay support. In the next step, the IgG fraction is isolated from a test sample, i.e., a sample undergoing examination, and the “test antibodies” thus obtained are detectably labeled. These labeled antibodies are then combined with an equal amount of “control antibodies” that have been isolated from a second sample of blood, serum or plasma. The control antibodies are attached to a second label that is different from and distinguishable from the label used for the test antibodies. The mixture of labeled test and control antibodies are incubated with the immobilized antigens and the relative amount of binding determined based upon the detectable labels. The assay procedure can be used to compare the antibodies present in patients having a disease such as cancer to the antibodies in samples from normal individuals. Results have indicated that the procedure can be used to identify antigens that are characteristic of prostate cancer, ovarian cancer and progressive benign prostate hyperplasia.

Detailed Summary

In its first aspect, the invention is directed to an assay for comparing the antibodies present in at least two samples of serum, blood or plasma. The assay involves first obtaining an immobilized array of antigens attached to a solid support by antibodies. The most important feature of these antibodies is that they recognize one particular antigen with a high degree of specificity. Thus, monoclonal antibodies and antibody fragments that retain their ability to bind antigen, e.g., Fab and F(ab)₂ fragments, can be used. In general, the antibody or antibody fragment should bind to the recognized antigen with at least a 100 fold greater affinity than to other antigens, with greater affinity being preferred. Solid supports will typically be either be glass or plastic slides or plates, although other types of immobile supports could potentially be used as well and will be understood to be equivalents.

The antigens bound to the immobilized antibodies are preferably obtained by lysing cultured cells or cells from tissues so as to retain the structural characteristics found in vivo. However, it is also possible to synthesize antigens or to use a combination of synthetic and natural antigens.

In the next step, test antibodies derived from a sample of blood, serum or plasma of a subject are obtained. The antibodies may be isolated using any procedure known in the art and should be attached to a detectable label, e.g., a dye. A second, control, group of antibodies is also obtained from a control sample of blood, serum or plasma. These are labeled with a second detectable label that can be distinguished from the first. Preferred labels are dyes or fluorescent compounds, with Cy3 and Cy5 fluorescent labels being most preferred. The main characteristic that must be maintained is that there must be some way for distinguishing the antibodies derived from the first and second samples, e.g., they should fluoresce or absorb at different wavelengths.

The labeled antibodies from the two samples are next mixed together (preferably in equal amounts) and incubated with the array of immobilized antigens. After the incubation, unbound antibody is removed from the array and the absorbance or fluorescence associated with each antigen is then determined. Since the specificity of the monoclonal antibodies originally attached to the plate is known, antigens that are differentially reactive to the host immune system, as reflected by a high degree of antibody binding, can be immediately identified.

The assay described above may be used to compare the antibody profiles of any two samples and will be of particular value in identifying profiles that are characteristic of disease states. For example, the blood, serum or plasma of a patient with a particular disease or condition may be compared with a one or more similar samples derived from a “control” source that is known to be free of the disease or condition. Antibodies that are present to a larger extent in samples derived from diseased individuals indicate that the antigens that they recognize are being produced to a greater extent when the disease is present. This knowledge will be of value to researchers studying the effects of the disease and, in some instances, may help in identifying potential therapies. In addition, antigens identified may be used diagnostically either individually or in combination. For example, the antigens characteristic of the presence of a particular disease may be immobilized on solid supports in the microarray assays described above or, alternatively, they may be quantitated using other assays.

Using the microarray assay described above, several antigens that are characteristic of the presence of prostate cancer have been identified and are shown in Table 3 and Table 6. These include: c-myc; MEK5; CLA-1 (CD36); SNK; p53; MUPP-1; 53BP2; and neurexen. Monoclonal antibodies recognizing at least one of the antigens in Table 3 or Table 6, and preferably several or all of them, should therefore be present in microarray assays for profiling antibodies. Some of these antigens do not appear to have previously been associated with prostate cancer and the present invention includes other types of diagnostic assays (e.g., radioimmunoassays or ELISA) that can be used to measure these antigens in serum or other samples. Particular antigens that are useful in this regard include: CLA-1 (CD36); SNK; MUPP-1; 53BP3; and neurexen. Assays for these antigens may be combined with other diagnostic tests for prostate cancer such as measurement of serum levels of prostate-specific antigen (PSA).

In the case of ovarian cancer, the antigen p90 ribosomal S6 kinase (RSK) is present to a greater extent in the serum of cancer patients. Thus, antibodies recognizing this antigen should be included among those immobilized on supports in microarray assays. Assays that specifically measure RSK, e.g., radioimmunoassays or ELISA assays, may also be used diagnostically.

Microarray profiling of antibodies can also be used for diseases or conditions other than cancer. For example, it has been found that the assay can be used to distinguish patients that have progressive BPH from those that have BPH that will not progress. The American Urological Association (AUA) uses a system for characterizing BPH based upon the severity of a variety of symptoms, e.g., frequency of urination (see J. Urol. 148:1549-1557 (1992)). Symptom scores can range from 0 (no symptoms) to 35 (severe symptoms). For the purposes of the present invention, progressive BPH is defined as disease that results in an increase in the AUA score of at least four points over a period of four years. Patients with progressive BPH, as opposed to patients with non-progressive BPH, have a serum antibody profile that is characterized by higher levels of antibodies recognizing the antigens: RB2 (Swiss protein accession no. Q08999); eEF2 kinase (Swiss protein accession no. 000418); cMyc (Swiss protein accession no. P01106); GM-CSF (Swiss protein accession no. P04141); CRIK (Swiss protein accession no. 014578); Ras (Ha) (Swiss protein accession no. Q9BR65); Cdk2 (Swiss protein accession no. P24941); NNOS (Swiss protein accession no. P29475); p73a (Swiss protein accession no. 015350); Tau-5 (Swiss protein accession no. P28595); and myogenin (Swiss protein accession no. P15173). Antibodies specifically recognizing these antigens should be included among those immobilized on supports for microarray assays. In addition, other types of assays, e.g., radioimmunoassays or ELISA assays, may be performed for the antigens. The presence of a high level of one or more of these antigens, when compared to a control (e.g., all patients with BPH, or patients known to have no BPH or non-progressive BPH) is an indication that the disease is likely to progress, and the treatment with drugs believed to retard progression (e.g., finasteride or doxazosin) is warranted.

The invention also includes glass or plastic plates or slides comprising monoclonal antibodies or antibody fragments attached to different sites which can be used by researchers or clinicians. At least one of the immobilized antibodies should recognize an antigen shown herein, including one or more of: c-myc; MEK5; CLA-1 (CD36); FNK; p53; MUPP-1; 53BP2; neurexen; RSK; B2 (Swiss Prot. accession no. Q08999); eEF-2 kinase (Swiss Prot. accession no. 000418); c-Myc (Swiss Prot. accession no P01106); GM-CSF (P04141); RSK; CRIK (Swiss Prot. accession no. 014578); Ras(Ha) (Swiss Prot. accession no. Q9BR65); Cdk2 (Swiss Prot. accession no. P24941); nNOS; (Swiss Prot. accession no. P29475); p73a (Swiss Prot. accession no. 015350); Tau-5 (Swiss Prot. accession no. P28595); and myogenin (Swiss Prot. accession no. P15173). The plates or slide may also optionally be prepared so that at least 90% of the immobilized antibodies are bound to antigen. The slides or plates may be used in methods for diagnosing whether a subject has a particular disease or condition such as prostate cancer, ovarian cancer or progressive BPH. The methods will follow the procedures discussed above in connection with microarray assays.

### BRIEF DESCRIPTION OF THE DRAWINGS

FIG. 1: This figure represents a schematic of a “reverse capture” microarray. Well-characterized, highly specific, and high affinity monoclonal antibodies are spotted on an array surface. Cell extracts containing the antigens are then immobilized to the respective spotted antibodies. This is then followed by incubation with labeled autoantibodies from a patient's serum. Test and control autoantibodies are then labeled with different CyDyes, and the ratio of the fluors determines the relative abundance of the autoantibodies in a given serum sample.

### DETAILED DESCRIPTION OF THE INVENTION

The present invention is concerned, inter alia, with the microarray profiling of serum antibodies as a method for identifying autoantibodies (and antigens) that are produced to a larger extent when a particular disease or condition is present. The Examples section describes studies involving prostate cancer, benign prostate hyperplasia and ovarian cancer, but the procedure should be applicable to other diseases and conditions as well. In fact, the method can be used for comparing the antibody profiles of any two individuals to determine the extent to which they may have been differentially exposed to antigens inducing an immune response. Although similar assays have been used in the past, these have generally employed an array of antigens that lack the secondary characteristics and structure found in vivo. In contrast, the present procedure may be used with antigens derived directly from cell lysates. In addition, by attaching the antibodies to surfaces using a monoclonal antibody, problems of denaturation are avoided.

The first step in the procedure involves immobilizing an array of monoclonal antibodies, each recognizing a specific antigen, to a surface such as a glass plate or slide. Monoclonal antibodies appropriate for use in such assays are commercially available, e.g., from Clontech and other manufacturers and in some cases it may be possible to purchase arrays already attached to a surface. If particular antigens are known to be associated with a disease or condition under examination, then monoclonal antibodies recognizing these antigens should be included in the array. If desired, fragments derived from the monoclonal antibodies that maintain the ability to specifically recognize antigen may also be used.

The next step in the procedure is to attach the antigens to the immobilized antibodies. This may be accomplished by lysing cells derived from culture or in vivo, removing cellular debris and then incubating the crude antigen solution with the array of immobilized antibodies. At the end of the incubation, unattached materials and antigens are removed, thereby leaving behind an array of antigens attached to slides or plates by the immobilized monoclonal antibodies. The identity of each of the attached antigens is known from the specificity of the antibody to which it is attach. In other words, each antibody is at a specific location on the slide or plate and recognizes only one particular type of antigen.

Once the array of immobilized antigens has been prepared, the next step is to prepare the antibody samples that will undergo testing. A sample of serum, plasma or blood is removed from a test subject. When assays are being used to identify antigens associated with a particular disease or condition, the test sample will be derived from a patient that has been diagnosed as having the disease or condition. A second “control” sample of blood, plasma or serum is then obtained from one or more other individuals that do not have the disease or condition. The IgG fraction present in the samples is then isolated using any method known in the art and the resulting antibodies are labeled. Any type of label that can be detected using a microarray assay is compatible with the present invention, with fluorescent dyes such as Cy3 and Cy5 being preferred. The main requirement for labeling is that the label attached to the antibodies derived from the test subject must be distinguishable from those derived from the control subject after binding has occurred. Thus, the absorption or emission wave lengths of the dyes should be sufficiently different to allow them to be readily distinguished. As described in the Examples section, this can be accomplished by labeling one set of antibodies with Cy5 and the other with Cy3.

After test and control antibodies have been labeled, an equal amount of each (e.g., 20 μg) is placed in a buffer solution and incubated with the array of immobilized antigens. The incubation buffer may consist of any type of standard buffer used in handling antibodies, e.g., PBS. The incubations may be carried out at about room temperature for a period ranging from 15 minutes to 2 hours with about 45 minutes being generally preferred. At the end of this time, unbound labeled antibody is removed and plates or slides are then analyzed to determine the amount of fluorescence or light absorption associated with each immobilized antigen. By comparing the results obtained using wavelengths characteristic of the dye attached to the test antibodies with those characteristic of the dye attached to the control antibodies, a profile can be obtained in which antibodies preferentially present in the test sample are identified. The presence of such antibodies is an indication that the antigens that they recognize are produced to a greater extent in the disease or condition present in the test subject.

Once it has been determined that a disease or condition is associated with a unique antibody profile, microarray assays can be used diagnostically to determine if the disease or condition is present. Antigens identified as being present in large amounts in the disease or condition should be included in the microarray profiles and the monoclonal antibodies recognizing these antigens should therefore be among those attached to the slides or plates. Alternatively, other types of assays that are specific for the particular antigens associated with a disease or condition may be performed. For example, once it is known that a particular antigen is associated with a disease, a radioimmunoassay or ELISA assay for the antigen or its autoantibody may be performed as a diagnostic procedure.

The microarray assay described herein has been used to identify a group of antigens that may help to diagnose patients with prostate cancer and to distinguish these individuals from those with other prostate conditions such as BPH. Another group of antigens has been found that can be used to distinguish progressive forms of BPH from forms that do not progress. Finally, the microarray assay has been used to identify a particular antigen that is associated with the presence of ovarian cancer. By applying the same methodology to other types of cancer and other types of disease conditions, additional antigens will be identified that are characteristic of those diseases and which may be used by researchers developing therapeutic and diagnostic procedures.

### EXAMPLES

### Example 1

### Reverse Capture Microarray Assays

The present example describes the development and use of a “reverse capture” antigen microarray, which allows the antigens to be immobilized in their native configuration, and facilitates the detection of autoantibodies in sera from patients with cancer. The assay can be used, inter alia, to distinguish patients with prostate cancer from patients with benign prostate disease.

A. Materials and Methods

Clinical specimens: Serum specimens were collected from patients with prostate cancer or benign prostate hyperplasia using approved informed consent forms. For patients with prostate cancer, serum was drawn prior to radical prostatectomy. For patients with benign prostate hyperplasia, serum was obtained during normal office visits. The clinical characteristics of the patients for prostate cancer and BPH are listed in Tables 1 and 2.

Cell culture and lysis: Two established human prostate cancer cell lines were used. LNCaP (androgen responsive) and PC3 (androgen independent) cells were obtained from the American Type Culture Collection (Rockville, Md.). Cells were cultured in RPMI 1640 with L-glutamine (Life Technologies, Inc., Grand Island, N.Y.), supplemented with 10% FBS, and 100 IU/ml penicillin and 100 μg/ml streptomycin. Cells were grown at 37° C. in a humidified atmosphere with 5% CO₂. Whole cell extracts were obtained by resolving cell pellets in Protein Extraction/Labeling Buffer (BD Biosciences Clontech, Palo Alto, Calif.). After rocking the suspension for 45 min at 4° C., the insoluble particulate fraction was removed by centrifugation (10 min at 4,500×g, 4° C.). The protein concentration was determined using a BCA Protein Assay Reagent Kit according to the manufacturer's instructions (Pierce Biotechnology, Inc., Rockford, Ill.).

Purification of IgG: IgG purification was performed using Melon™ Gel IgG Purification Kit (Pierce Biotechnology, Inc., Rockford, Ill.) according to the manufacturer's protocol. IgGs were adjusted to a concentration of 2 mg/ml in PBS supplemented with 0.1% (w/v) sodium azide and stored at 4° C. until use. The purity of the IgGs was determined by running aliquots onto 8% SDS-PAGE gels.

Differential fluorescent labeling of IgGs: Purified IgGs were labeled with monofunctional Cy3 and Cy5 fluors (Amersham Biosciences, Corp., Piscataway, N.J.). Fifty micrograms of IgGs were labeled by working fluor solution by vortexing in a microfuge tube for one minute. The samples containing the CyDye were then allowed to react by placing the samples on ice for 90 minutes in the dark. The labeling reaction was then stopped by the addition 4 ul of Blocking Buffer. Unbound Dye was removed using Protein Desalting Spin Columns (all described in detail in the Antibody Microarrays User Manual, BD Biosciences Clontech, Palo Alto, Calif.).

Antibody microarray: BD Clontech AB Microarray 500 was used to immobilize the antigens. The array consists of 500 distinct, well-characterized monoclonal antibodies. All 500 arrayed antibodies are carefully tested for specificity and sensitivity. Those that display a high degree of cross-reactivity are eliminated from the final product. A wide variety of proteins (both cytosolic and membrane-bound), representing a broad range of biological functions, can be detected by the antibody microarray. Targets include proteins involved in signal transduction, cell-cycle regulation, gene transcription, apoptosis, cell growth, and oncogenesis. A complete list of the arrayed antibodies, including Swiss-Prot ID numbers of the target antigens, is available from the manufacturer (see http://bioinfo2.clontech.com/abinfo/array-list-action.do).

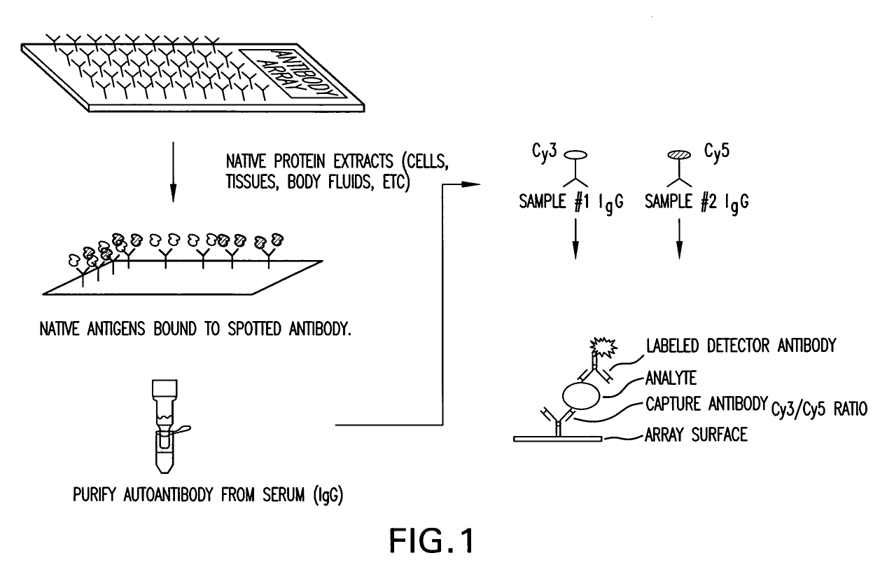

Native protein array production and probing: Mixed PC3 and LNCaP cell extracts (250 ug) were placed in 5 ml of Incubation Buffer with AB Microarray 500 slides and incubated for 45 min at room temperature with gentle rocking. Array slides were washed three times with PBS and incubated with equal amounts (20 ug) of CyDye labeled IgGs for 45 min at room temperature. The arrays were washed again, spin-dried, and scanned with a ScanArray 4000XL scanner as described in the Antibody Microarrays User Manual (BD Biosciences Clontech, Palo Alto, Calif.). A schematic of the “reverse capture” microarray is illustrated in FIG. 1.

Validation of Antigen-Autoantibody Reactivity in Individual Serum Specimens with Immunoprecipitation and Western Blot Analysis

Antibodies (IgG) were isolated from 50 ul of serum from individual patients using the Melon™ Gel IgG Purification Kit (Pierce Biotechnology, Inc., Rockford, Ill.). Cell extracts from human prostate cancer cell lines LNCaP and PC-3 were mixed at a 1:1 ratio for immunoprecipitation. Immunoprecipitation and elution of antigens was performed with the Protein G Immunoprecipitation Kit from Sigma-Aldrich Corp., St. Louis, Mo., as described by the manufacturer. Following immunoprecipitation and elution, the precipitated antigens were quantified using a DC Protein Assay Kit (BioRad Laboratories, Hercules, Calif.), and prepared for Western blot analysis. Equal amounts (25 ug per lane) of eluted antigens were separated on 8-16% linear gradient polyacrylamide gels and transferred to nitrocellulose membranes. Non-specific interactions were blocked with 5% milk in TBS, followed by the addition of mouse monoclonal antibody against 53BP2 or MUPP1 (BD Biosciences Pharmingen, San Diego, Calif.). 53BP2 and MUPP1 were chosen for validation as examples based on the results of our study (see Results section). Secondary antibody conjugated with horseradish peroxidase was then added, and the band visualized with an enhanced chemilluminescence system (ECL, Amersham Biosciences Corp., Piscataway, N.J.).

Data Analysis

The raw data were first normalized via a two-stage ANOVA technique (Lee, et al. J. Biopharm. Stat. 12:1-19 (2002); Lee, Analysis of Microarray Gene Expression Data, Kluwer Academic Publishers, Boston (2004); Wolfinger, et al., J. Comput. Biol. 8:625-637 (2001)) to separate the effect of sample type from other effects, such as dye, slide, and replication effects. The significant antigens were then selected based on the normalized antigen abundances by using an interquartile method (Lee, et al. J. Biopharm. Stat. 12:1-19 (2002); Lee, Analysis of Microarray Gene Expression Data, Kluwer Academic Publishers, Boston (2004)). The two dye-reversed experiments were analyzed separately because the subject sera pools in the two experiments are different. The final list of significant antigens is the union of the two significant antigen lists of the two experiments. The union of the normalized antigen abundances of the two experiments was used to perform two-way clustering analyses. STATA was used to perform the two-stage ANOVA normalization and antigen selection. JMP was used to perform the two-way clustering analyses and to prepare the heat map. The clustering analyses used the Ward hierarchical clustering method.

B. Results

Antibody microarray and cell extracts: FIG. 1 is a schematic of the “reverse capture” autoantibody microarray. This microarray is based on the dual-antibody sandwich immunoassay of ELISA. The basic platform is 500 highly specific, high affinity monoclonal antibodies that were spotted onto a glass slide. These monoclonal antibodies were then used to immobilize their corresponding native antigens. Using the immobilized antigens as “baits,” we compared antigen-autoantibody reactivity. Since our goal was to use the antibodies to immobilize specific antigens as “baits” for autoantibody profiling, we first tested the number of targets that could potentially be identified on the array. Using combined cell extracts from human prostate cancer LNCaP and PC-3 cells, proteins were labeled with Cy3 dye and hybridized onto the microarray slide. It was found that nearly all (˜90%) of the antigens, as determined by the 500 monoclonal antibodies on the array, can be identified when using the combined cell extracts.

Preferential reactivity of autoantibodies from sera of patients with prostate cancer versus benign prostate hyperplasia (BPH): A major need in clinical management of prostate diseases is to identify potential biomarkers that can distinguish prostate cancer from BPH. Using the immobilized antigens as “bait,” we tested the feasibility of autoantibody profiling as a potential strategy to distinguish prostate diseases. IgG from patients with prostate cancer was labeled with Cy3 dye, and IgG from patients with documented BPH was labeled with Cy5 dye. Results of incubation with antibody microarrays showed clearly preferential reactivity to the immobilized antigens between prostate cancer patients and patients with BPH. In addition, there is a consistent preferential reactivity to the immobilized antigens in different patients with prostate cancer. Since the AB Microarray is spotted with known monoclonal antibodies, the antigens can be easily identified. For example, antigens such as c-Myc, p53, IL-1b, neurexin I are consistently detected by autoantibodies from patients with prostate cancer. A list of these antigens for prostate cancer is shown in Table 3.

Since our “reverse capture” autoantibody microarray is dependent on the labeling of the autoantibodies, we addressed whether other proteins from the sera are present following IgG purification which might interfere with our reading. We determined the purity of our autoantibodies by loading an aliquot of the purified antibody from each serum onto 8% SDS-PAGE gels. Only two bands, corresponding to the heavy and light chains of IgG, are visible following IgG purification.

Control for variability in labeling efficiency: To control for potential variability in labeling efficiency, we repeated the autoantibody reactivity by interchanging the CyDyes. IgG from a patient with prostate cancer was first labeled with Cy3 dye, and then repeated with Cy5 dye. Although there are dye intensity differences on specific antigens with different CyDyes, many of the antigen-autoantibody reactivities are similar. However, to take into consideration the CyDye labeling efficiencies and control for differences in autoantibody binding efficiencies following labeling, we performed and analyzed our data using a two-slide dye “swap” protocol for each sample group. With this protocol, each group of samples generated four readings: prostate cancer (Cy3), BPH (Cy5), prostate cancer (Cy5), and BPH (Cy3). These four samples were used to form two mixes. Each of these mixes was then incubated with a “reverse capture” microarray. In this set-up, Slide 1 measured Prostate Cancer (Cy3)/BPH (Cy5), while Slide 2 measured Prostate Cancer (Cy5)/BPH (Cy3). Once the slides were scanned, the ratios from each slide were then analyzed. Using two-dimensional hierarchical clustering from dye intensity data with the reverse-color array pairs, specific differential autoantibody reactivity between prostate cancer and BPH could be clearly identified.

Validation of potential antigen-autoantibody reactivity: To confirm and to validate our antigen-autoantibody reactivity, we isolated autoantibodies from individual serum specimens. The autoantibodies were used to immunoprecipitate the antigens from a reference cell extract using a Protein G Immunoprecipitation Kit from Sigma-Aldrich Corp., St. Louis, Mo. Following immunoprecipitation, the antigens were eluted from the Protein G slurry, quantified, and loaded onto a gel for Western blot analysis. The probe used for Western blot detection was the monoclonal antibody corresponding to the candidate antigen that was immobilized on the “reverse capture” autoantibody microarray. Two putative “biomarkers” were identified as significant from two-dimensional hierarchical clustering from dye intensity data with our two-slide dye “swap” protocol. Using monoclonal antibody to 53BP2, we demonstrated that 7 out of 9 of our prostate cancer serum samples contained autoantibody against 53BP2 and 0 out of 9 of our BPH serum samples contained autoantibody against 53BP2. Similarly, using monoclonal antibody to a multi-PDZ domain protein MUPP1, we demonstrated that 9 out of 10 of our prostate cancer serum samples contained autoantibody against MUPP1 and only 4 out of 10 of our BPH serum samples contained autoantibody against MUPP1.

TABLE 1
Clinical information on prostate cancer serum specimens

| | Age | PSA | | TNM |
|---|---|---|---|---|
| Sample no. | (years) | (ng/ml) | Gleason grade | status |
| 1 | 57 | 4.77 | 3 + 3 | T1c |
| 2 | 58 | 4.3 | 4 + 3 | T2c |
| 3 | 54 | 1.0 | 3 + 3 | T2b |
| 4 | 63 | 7.1 | 3 + 3 | T2c |
| 5 | 67 | 5.2 | 3 + 4 | T1c |
| 6 | 56 | 2.35 | 3 + 3 | T2c |
| 7 | 50 | 5.3 | 3 + 3 | T2c |
| 8 | 58 | 5.1 | 3 + 3 | T1c |
| 9 | 68 | 4.6 | 4 + 3 | T1c |
| 10 | 53 | 4.8 | 3 + 3 | T1c |

PSA: prostate specific antigen;
TNM: tumor-node-metastasis classification system.
Mean PSA is 4.45 ng/ml.
Mean age is 58.4 years old.

TABLE 2
Clinical information on benign prostate hyperplasia serum
specimens

| | | | symptom score |
|---|---|---|---|
| Sample no. | Age (years) | PSA (ng/ml) | (AUA) |
| 1 | 71 | 9.5 | 12 |
| 2 | 68 | 4.7 | n.d. |
| 3 | 58 | 4.7 | 10 |
| 4 | 78 | 3.5 | 1 |
| 5 | 57 | 4.0 | n.d. |
| 6 | 51 | 5.2 | 22 |
| 7 | 55 | 4.1 | n.d. |
| 8 | 75 | 6.1 | n.d. |
| 9 | 73 | 3.1 | 24 |
| 10 | 62 | 1.1 | 10 |

n.d.: not determined;
PSA: prostate specific antigen;
Mean PSA is 4.6 ng/ml.
Mean age is 64.8 yrs old.
Scores for the American Urological Association (AUA) symptom score can range from 0 (no symptoms) to 35 (severe symptoms). J. Urol. 148: 1549-1557 (1992).

TABLE 3
Elevated proteins in prostate cancer serum as detected by the
“reverse capture” antigen microarray

| Clontech | SwissProt | Protein/Antigen |
|---|---|---|
| Antibody | Accession | Description |
| AB_000664 | Q9H4B4 | Cytokine-inducible serine/threonine-protein |
| | | kinase |
| AB_001253 | P01584 | Interleukin 1, beta |
| AB_000243 | P29475 | Nitric oxide synthase 1 (neuronal) |
| AB_001264 | Q13625 | Tumor suppressor p53-binding protein 2 |
| AB_000837 | P01106 | Myc proto-oncogene protein |
| AB_001200 | O75970 | Multi PDZ domain protein MUPP1 |
| AB_000314 | O60610 | Diaphanous protein homolog 1 |
| AB_000968 | P23025 | DNA-repair protein complementing XP-A cells |
| AB_001171 | O14578 | citron (rho-interacting, serine/threonine kinase |
| | | 21) |
| AB_001237 | Q01085 | TIA1 cytotoxic granule-associated RNA |
| | | binding protein-like 1 |
| AB_000993 | Q14209 | Transcription factor E2F2 |
| AB_000612 | Q07864 | DNA polymerase epsilon, catalytic subunit A |
| AB_001211 | O95196 | Chondroitin sulfate proteoglycan 5 |
| | | (neuroglycan C) |
| AB_000611 | Q92499 | DEAD (Asp-Glu-Ala-Asp) box polypeptide 1 |
| AB_000513 | Q07817 | BCL2-like 1 |
| AB_000390 | P23443 | Ribosomal protein S6 kinase, 70 kDa, |
| | | polypeptide 1 |
| AB_000565 | Q9BQH3 | Calcium/calmodulin-dependent protein kinase |
| | | kinase 1, alpha |
| AB_000757 | P05455 | Sjogren syndrome antigen B (autoantigen La) |
| AB_000545 | P14923 | Junction plakoglobin |
| AB_000562 | P27824 | Calnexin |
| AB_001128 | O76043 | Protein tyrosine phosphatase, receptor-type, Z |
| | | polypeptide 1 |
| AB_000578 | Q14016 | Scavenger receptor class B, member 1 |
| AB_001203 | Q9ULB1 | Neurexin 1-alpha precursor |
| AB_001273 | Q00403 | Transcription initiation factor IIB |
| AB_000318 | P19878 | Neutrophil cytosol factor 2 |
| AB_000897 | P10636 | Microtubule-associated protein tau |
| AB_000373 | Q13094 | Lymphocyte cytosolic protein 2 |
| AB_001181 | Q12828 | Far upstream element (FUSE) binding protein 1 |
| AB_000306 | P31323 | Protein kinase, cAMP-dependent, regulatory, |
| | | type II, beta |
| AB_000284 | P17252 | Protein kinase C, alpha |
| AB_000788 | P28482 | Mitogen-activated protein kinase 1 |
| AB_001189 | O60620 | Katanin p80 (WD repeat containing) subunit |
| | | B1 |
| AB_001147 | P01375 | Tumor necrosis factor (TNF superfamily |
| | | member 2) |
| AB_001225 | P19525 | Interferon-induced, double-stranded RNA- |
| | | activated protein kinase |
| AB_000675 | Q9NQX3 | Gephyrin |
| AB_000294 | P24723 | Protein kinase C, eta |
| AB_001292 | Q9Y218 | Optineurin |
| AB_000523 | Q07814 | BCL2-associated X protein |
| AB_001116 | Q9NQ66 | Phospholipase C, beta 1 (phosphoinositide- |
| | | specific) |
| AB_000603 | P52824 | Diacylglycerol kinase, theta |
| AB_001012 | P30307 | CDC25C |
| AB_000971 | P49662 | Caspase 4, apoptosis-related cysteine protease |
| AB_000293 | P04637 | Cellular tumor antigen p53 |
| AB_000271 | Q15233 | Non-POU domain containing, octamer-binding |
| AB_001173 | O43602 | Doublecortin |
| AB_000953 | P07385 | CrmA, Serine proteinase inhibitor 2 |
| AB_001198 | O96018 | Amyloid beta A4 precursor protein-binding |
| | | family A member 3 |
| AB_000549 | P30281 | G1/S-specific cyclin D3 |

### Example 2

### Differential Reactivity of Autoantibodies from Cancer and BPH Patients

Separate experiments were also performed in which serum from individual cancer patients was individually tested against pooled serum from BPH patients. (Note that in Example 1, both the sera of cancer patients and the sera of BPH patients were pooled prior to analysis.) This resulted in the identification of several additional markers (i.e., besides those in Table 3) which may be used to distinguish cancer patients from BPH patients (i.e., there is a greater elevation of autoantibodies to these antigens in cancer patients than in BPH patients).

Tables 4 and 5 present clinical data of the patients whose sera was used to determine significant antigens, i.e. antigens to which significant differential autoantibody reactivity among prostate cancer patients was determined, in a series of six experiments in which each individual prostate cancer patient's serum was tested for differential autoantibody reactivity against autoantibodies from BPH serum belonging to a group of 5 individual patients; equal amounts of antibody were contributed by each BPH patient.

TABLE 4
Clinical information on prostate cancer serum specimens

| | Age | PSA |
|---|---|---|
| Sample no. | (years) | (ng/ml) |
| 41 | 59 | 7.9 |
| 47 | 67 | 5.2 |
| 50 | 58 | 5.1 |
| 56 | 63 | 6.3 |
| 74 | 62 | 5.4 |
| 76 | 69 | 7.9 |

PSA: prostate specific antigen;
Mean PSA is 6.3 ng/ml.
Mean age is 63 years old.

TABLE 5
Clinical information on benign prostate hyperplasia serum
specimens

| Sample no. | Age (years) | PSA (ng/ml) |
|---|---|---|
| 42 | 71 | 9.5 |
| 47 | 68 | 4.7 |
| 121 | 57 | 4.0 |
| 122 | 51 | 5.2 |
| 125 | 75 | 6.1 |

PSA: prostate specific antigen;
Mean PSA is 5.9 ng/ml.
Mean age is 64.4 yrs old.

Table 6 shows significant antigens that were found in a series of six experiments in which each individual prostate cancer patient's serum was tested for differential autoantibody reactivity against autoantibodies from BPH serum belonging to a group of 5 individual patients; equal amounts of antibody were contributed by each BPH patient.

TABLE 6
Additional Elevated proteins in prostate cancer serum

| | | |
|---|---|---|
| AB_000559 | P55060 | Cellular apoptosis susceptibility protein |
| AB_001165 | Q12873 | Chomodomain helicase-DNA-binding protein 3 |
| AB_000608 | Q08495 | Dematin |
| AB_000643 | P54687 | Branched-chain-amino-acid aminotransferase, |
| | | cytosolic |
| AB_000733 | P17936 | Insulin-like growth factor-binding protein 3 |
| | | (precursor) |
| AB_000569 | Q05682 | L-Caldesmon |
| AB_001279 | P07099 | Epoxide hydrolase 1 |
| AB_001032 | Q13469 | Nuclear factor of activated T-cells, |
| | | cytoplasmic 2 |
| AB_000868 | P17947 | Transcription factor PU.1 |
| AB_000380 | Q13485 | Mothers against decapentaplegic homolog 4 |
| AB_000397 | P29597 | Non-receptor tyrosine-protein kinase TYK2 |
| AB_000477 | Q9UBV9 | Tumor protein p73-like |
| AB_000977 | P24385 | G1/S-specific cyclin-D1 |
| AB_001320 | P04637 | Cellular tumor antigen p53 |
| AB_001102 | P08567 | Pleckstrin |
| AB_001254 | P08700 | Interleukin-3 (precursor) |

### Example 3

### Reverse Capture Microarray Assays for BPH

There have been some indications that BPH may induce detectable changes in serum antibodies (Anim, et al., Acta Histochem. 100(4):439-449 (1998); Nickel, et al., B. J. Urol. Int. 84(9):976-981 (1999); Di Silverio, et al. Eur. Urol. 43(2):164-175 (2003); Mahapokai, et al., Vet. Immunol. Immunopathol. 78(3-4):297-303 (2001); Prakash, et al., Proc. Nat'l Acad. Sci USA 99(11):7598-7603 (2002)). In the present example, the reverse capture microarray assay was used in an attempt to identify characteristic antigen-autoantibody reactivity in patients with this condition. The results suggest that the serum autoantibody repertoire from patients with BPH can be used to predict disease outcome. In addition, the identified antigens may serve to define aberrant cellular mechanisms for disease progression, and as potential targets for therapy and/or prevention (Tan, J. Clin. Invest. 108:1411-1415 (2001)).

### Results and Discussion

Development and proof-of-principle of our “reverse capture” autoantibody microarray: Assays were performed using the BD Clontech AB Microarray 500 as described in Example 1. We first tested the number of targets that can potentially be identified on the array. Combined cell extracts (250 μg each) from human prostate cancer LNCaP and PC-3 cells were labeled with Cy3 dye (green, Amersham Biosciences Corp., Piscataway, N.J.) and hybridized onto the AB Microarray slide. It was found that nearly all (about 90%) of the antigens, as determined by the 500 monoclonal antibodies on the array, can be immobilized when using the combined cell extracts.

Preferential autoantibody reactivity in patients with BPH: We also determined whether there is any differential autoantibody reactivity between samples obtained in the MTOPS (Medical Therapy of Prostatic Symptoms) Study from BPH patients with clinical progression and samples from patients without clinical progression. For our purpose, BPH progression is defined as “greater than or equal to a 4-point increase in the baseline AUA symptom score” within the shortest possible time period following initial enrollment. ALL samples were matched for baseline PSA and baseline prostate volume. Sample selection criteria were: PSA levels greater than or equal to 3.3 ng/ml with prostate volumes greater than or equal to 40 cm³ who progressed (N=9) versus those with similar baseline characteristics that did not progress by year-4 of the MTOPS trial (N=9) (defined as AUA symptom score of less than a 4-point increase in the baseline by year-4 of the MTOPS trial). For a detailed clinical characteristics of the samples used, please see Table 7.

Antibodies from BPH patients with clinical progression were labeled with Cy3 dye (green), and patients without progression were labeled with Cy5 dye (red). In addition, the CyDyes were reversed, i.e., antibodies from BPH patients with clinical progression were labeled with Cy5 dye (red), and patients without progression were labeled with Cy3 dye (green). After allowing hybridization to the microarray slide, it was found that there is differential autoantibody reactivity between the two groups. Several antigens that were preferentially expressed in the BPH progression group include well-known proteins involved in cell cycle or proliferation events, e.g., Rb2, eEF-2 kinase, cyclin D1, cyclin D3, Ha-ras, and proliferating cell nuclear antigen (PCNA). In addition, we have identified a Citron Rho-interacting kinase, CRIK, which appears be involved in the regulation of contractile activity by regulating myosin light chain phosphorylation (Di Cunto, et al. J Biol. Chem. 273:29706-29711:1998; Yamashiro, et al., Mol. Biol. Cell 14:1745-1756 (2003)). A list of antigens preferentially expressed in BPH patients whose disease progresses is shown in Table 8.

The results obtained are consistent with published data derived from expression microarrays of tissues between symptomatic and asymptomatic BPH. Parkash et al. (Proc. Nat'l Acad. Sci. 99(11):7598-7603 (2002)) demonstrated that genes associated with cell proliferation (e.g., calcium/calmodulin-dependent serine kinase, S-phase kinase-associated protein 2 or p45) were significantly up-regulated in the symptomatic BPH group.

TABLE 7
Clinical characteristics of BPH serum specimens

| | |
|---|---|
| No AUA Progression | AUA Progression |
| (>4 yrs) | (<2.5 yrs) |

| Sample # | PSA | Volume | PSA | Volume |
|---|---|---|---|---|
| 1 | 6.4 | 95.3 | 4.7 | 78.6 |
| 2 | 6.8 | 73.3 | 3.8 | 50.4 |
| 3 | 4.4 | 59.0 | 5.0 | 64.1 |
| 4 | 3.5 | 82.0 | 4.4 | 76.8 |
| 5 | 4.1 | 47.0 | 5.2 | 61.4 |
| 6 | 4.7 | 59.5 | 5.8 | 58.9 |
| 7 | 7.5 | 62.0 | 6.7 | 46.1 |
| 8 | 8.2 | 60.0 | 5.9 | 50.0 |
| 9 | 5.5 | 42.8 | 3.5 | 50.0 |
| 10 | 4.6 | 44.6 | | |
| 11 | 4.7 | 74.3 | | |
| 12 | 3.5 | 61.2 | | |
| 13 | 5.5 | 67.1 | | |
| 14 | 6.9 | 58.8 | | |
| 15 | 3.8 | 41.0 | | |
| 16 | 4.3 | 70.0 | | |
| 17 | 4.7 | 70.0 | | |
| 18 | 9.9 | 64.5 | | |

Mean PSA: No progression: 5.68 ng/ml (#1-9); 5.32 ng/ml (#10-18). Progression: 5.0 ng/ml.
Mean prostate volume: No progression: 64.54 cc (#1-9); 61.28 cc (#10-18). Progression: 59.59 cc.

TABLE 8
Markers of BPH progression

| SwissProt Accession # | Antigen name |
|---|---|
| Q08999 | Rb2 |
| O00418 | eEF-2 kinase |
| P01106 | c-Myc |
| P04141 | GM-CSF |
| O14578 | CRIK |
| Q9BR65 | Ras (Ha) |
| P24941 | Cdk2 |
| P29475 | nNOS |
| O15350 | p73a |
| Q28595 | Tau-5 |
| P15173 | myogenin |

### Example 4

### Example Assay Protocol

The present example provides a microarray protocol that could be used for serum antibody profiling.

A. Materials

Monoclonal Antibodies

An array of monoclonal antibodies recognizing known antigens may be purchased commercially. In the present example, the BD Clontech™ Ab Microarray containing two copies of each of 500 different antibodies immobilized on glass slides is used. The slides with the immobilized antibodies are supplied in Storage Buffer inside a capped Storage Vial. The kit also contains an incubation tray and several different buffers: 20 ml Extraction/Labeling Buffer; 200 μl Blocking Buffer; 20 ml 10× Desalting Buffer; 90 ml Stock Incubation Buffer; 10 ml Background Reducer; 20 ml Wash Buffer 1; 20 ml Wash Buffer 2; 20 ml Wash Buffer 3; 20 ml Wash Buffer 4; 20 ml Wash Buffer 5; 20 ml Wash Buffer 6; and 20 ml Wash Buffer 7.

Additional Materials and Equipment

BCA Protein Assay Reagent Kit (Pierce Biotechnology; Cat. No. 23225 or 23227) provides a detergent-compatible BCA reagent for quantifying total protein and bovine serum albumin for use as a protein standard. Pierce's BCA Protein Assay Reagent Kit may be used for all Ab Microarray analyses, unless noted otherwise.

DyLight™ 547 and DyLight™ 647 Monoclonal Antibody Labeling Kits (Pierce Biotechnology; Cat. No. 53009 and 53015). DyLight™ 547 and 647 are fluorescent dyes that have distinct emission spectra. Pierce Biotechnology supplies the DyLight™ 547 and 647 dyes as monofunctional N-hydroxysuccinimide (NHS)-esters in dried pre-measured amounts. The NHS-ester is a functional group that reacts with primary amines. The reaction produces a covalent bond, which links the dye to the amine. Each labeling kit contains: DyLight 547 or 647 NHS Ester, 5×20 μg vials; Dimethylformamide, 2 ml; Borate buffer (0.67 M), 1 ml; Zebra™ Desalt Spin Columns, 10×0.5 ml columns.

Melon™ Gel Kit (Pierce Biotechnology; Cat. No. 45206). Kit Contents: Melon™ Gel IgG Purification Support, 3 ml of settled gel, supplied as a 20% slurry (i.e., 15 ml total volume); Melon™ Gel Purification Buffer, 100 ml; Handee™ Mini-Spin Columns and accessories.
0.5-ml, 1.5-ml and 2.0-ml microcentrifuge tubes.
15-ml and 50-ml centrifuge tubes (e.g., BD Falcon™ conical centrifuge tubes)
Protein Desalting Spin Columns (Piercee Biotechnology, Cat. No. 89849 or 89862).
Alumina.
Mortar and pestle.
Disposable PD-10 Desalting Column (Amersham Biosciences, Cat. No. 17-0851-01).
M sodium carbonate buffer (pH 8.3).
Phosphate buffered saline (PBS) (pH 7.4).
Ultra-pure water.
Rocking platform
End-over-end rotator.
Pipettes.
Pipette tips.
Laboratory tissues.
Swinging-bucket centrifuge (with adaptors for spinning 50-ml tubes).
Microcentrifuge.
96-well plates.
Spectrometer capable of measuring absorbance at 562 and 750 nm.
Microarray scanner compatible with 75×25×1 mm slides and capable of dual-color analysis. The scanner must be capable of measuring fluorescence in the range of the Cy3 and Cy5 fluorescent labels.
Microarray scanning software.
Microsoft Excel.
Microarray data analysis software.

B. Methods

Extracting Native Protein from Cells
1. Wash 2 150-mm cell culture plates of 90% confluent cells four times with 20 volumes of PBS (pH 7.4). This should yield about 75 mg of cells.
2. Use a cell scraper or cell lifer to harvest the cells and place the cells in a pre-weighed microcentrifuge tube.
3. Centrifuge the harvested cells at 4,000×g for 3 min to pellet the cells.
4. Decant the supernatant and aspirate the residual liquid.
5. Centrifuge the tube again for 2 min at 4,000×g and aspirate any residual traces of liquid, then reweigh the tube to determine the weight of the cell pellet.
6. Freeze the cells by placing the tube in a −80° C. freezer for about 45 min.
7. Place the cells at room temperature, and add 20 μl of Extraction/Labeling Buffer for each mg of cells.
8. Mix thoroughly by vortexing. Check to be sure the mixture is homogeneous.
9. Incubate the samples at room temperature for 10 min with constant end-over-end rotation.
10. Centrifuge the suspension at 10,000×g for 30 min at 4° C.
11. Transfer the supernatant to a clean tube (discard the pellet).
12. Measure the protein concentration using Pierce's BCA™ Protein Assay Reagent Kit.
13. Dilute the sample to 2 mg protein/ml by adding the appropriate volume of Extraction/Labeling Buffer. The final volume should be at least 400 μl.
14. Proceed immediately with the Antibody Array Incubation.

Extracting Native Protein from Crude Tissue

Before beginning this section of the protocol, chill the following items on ice or at 4° C.:
Extraction/Labeling Buffer
one mortar and pestle
two 2 ml microcentrifuge tubes
1. Transfer 15-25 mg of frozen tissue to the pre-chilled mortar.
2. Add 2-5 mg of alumina to the mortar.
3. Use the pestle to grind the tissue until a paste is formed.
4. Add 100-200 μl of pre-chilled Extraction/Labeling Buffer to the mortar.
5. Mix the buffer into the paste using the pestle. When finished, use a micropipette tip to scrape any paste adhered to the pestle back into the mortar.
6. Transfer the extract to a pre-chilled 1.5 or 2 ml microcentrifuge tube.
7. Holding the pestle over the mortar, rinse the pestle with 100-200 μl of Extraction/Labeling Buffer.
8. Combine the rinse from Step 7 with the original extract in the 2 ml tube. (Use the second pre-chilled microcentrifuge tube if the volume exceeds the first tube's capacity.)
9. Centrifuge the suspension at 10,000×g for 30 min.
10. Carefully transfer the supernatant to a pre-chilled 1.5 or 2 ml microcentrifuge tube, taking care not to disturb the pellet.
11. Gently invert the tube to mix the lysate.
12. Measure the protein concentration using Pierce's BCA™ Protein Assay Reagent Kit.
13. Dilute each sample to 2 mg protein/ml by adding the appropriate volume of Extraction/Labeling Buffer. The final volume should be at least 400 μl.
14. Proceed immediately with the Antibody Array Incubation.

Extracting Native Protein from Body Fluid

This section of the procedure can also be carried out on a smaller-scale if the body fluid being used has a high concentration of protein. However, at least 400 μl of protein will be needed at a concentration of 2 mg/ml after desalting. A single Protein Desalting Spin Column, by Pierce, is appropriate for preparing native protein from body fluid sample volumes of up to 80 μl.

As long as the following steps are done quickly, they can be completed at room temperature. Otherwise, if access to a cold room is available, the procedure can be completed at 4° C.
1. Using Pierce's BCA™ Protein Assay Reagent Kit, measure the concentration of protein in the body fluid you will be using. If your protein source is urine, you should use Bio-Rad's RC DC Protein Assay Kit (Bio-Rad Laboratories, Cat. No. 500-0119) to determine the concentration of protein in your sample (the BCA™ kit is incompatible with the uric acid found in urine).
2. Dilute or concentrate your sample to around 4 mg protein/ml. If you must dilute the sample, use 0.1 M sodium carbonate buffer (pH 8.3).
3. Equilibrate the PD-10 column with 3×5 ml of 0.1 M sodium carbonate buffer (pH 8.3).
4. Load 2.5 ml of your sample (˜4 mg/ml) onto the column.
5. Once the sample has passed into the column, place a fresh 15 ml conical centrifuge tube under the column.
6. Add 3.5 ml of 0.1 M sodium carbonate buffer (pH 8.3).
7. Using Pierce's BCA™ Protein Assay Reagent Kit, measure the protein concentration in the eluted solution.
8. Dilute the sample to 2 mg protein/ml be adding the appropriate volume of Extraction/Labeling Buffer.
9. Proceed immediately with the antibody array incubation.

Antibody Array Incubation with Native Antigens
1. Set up the Incubation Tray provided. Note that it contains four separate chambers for incubating and washing microarray Slides 1 and 2. Mark the exterior surface of the tray with a pen as a reminder of these assignments: Slide 1 Incubation, Slide 1 Wash, Slide 2 Incubation, Slide 2 Wash
2. Prepare 50 ml Incubation Buffer from 45 ml Stock Incubation Buffer and 5 ml Background Reducer.
3. Add 5 ml of Incubation Buffer to the incubation chambers.
4. Transfer 200 μg of protein (2 mg/ml) to each of the incubation chambers.
5. Incubate the tray at room temperature for 30 min with gentle rocking.
6. Meanwhile, prepare the Ab Microarrays by washing the slides two times as follows:
   a. While pressing a gloved finger against the top of the vial to keep the slides from falling out, decant the Storage Buffer from the Storage Vial.
   b. Add 30 ml of Stock Incubation Buffer.
   c. Cap the Storage Vial. Then slowly invert the vial 10 times.
   d. Decant the Stock Incubation Buffer while using a gloved finger to keep the slides from falling out.
   e. Add 20 ml of Incubation Buffer.
   f. Repeat steps c and d.
   g. Stand the vial upright in a rack.
7. Record each slide's lot number.
8. Remove the slides one-by-one from the Storage Vial and place each one, array side-up, in the incubation chamber containing the Incubation Buffer/Antigen Mix to which it has been assigned.
9. Incubate the slides at room temperature for 45 min with gentle rocking. Every 10 min, perform the following manipulation to assist the exchange of liquid on all sides of the slide: Use a pipette tip to pry up one end of the slide while gently rocking the Incubation Tray once or twice.
10. Remove the buffer from the incubation chambers.
11. Add 5 ml of 1×PBS (pH 7.4) to the incubation chambers.
12. Incubate at room temperature for 5 min with gentle rocking.
13. Repeat Steps 11-12 two times.
14. After washing, the native protein array is ready for incubation with labeled sample antibodies.

Purification of IgG Using Melon Gel Kit

The following steps may be carried out several days prior to the rest of the experiment. Purified IgG can be stored for a few days at 4° C. If IgG is to be stored for longer than a few days, aliquots should be placed in a −20° C. freezer for storage until use. Repeated freeze/thaw cycles should be avoided.
1. Equilibrate the Melon™ Gel IgG Purification Support and Purification Buffer to room temperature (about 30 min).
2. Swirl the bottle containing the Purification Support (do not vortex) to obtain an even suspension. To ensure proper gel slurry dispensing, use a wide bore or cut pipette tip to dispense 500 μl of gel slurry into a Handee™ Mini-Spin Column placed in a microcentrifuge tube. Swirl the bottle of gel slurry before pipetting each sample to maintain the gel suspension.
3. Centrifuge the uncapped column/tube assemblies for 1 minute at 3,000×g, then remove the spin columns and discard flow-through.
4. Add 300 μl of Purification Buffer to the column, pulse centrifuge for 10 seconds and discard flow-through. Repeat this wash twice. Place the bottom caps on the columns.
5. Add 50 μl of each serum sample diluted 1:10 in 1× Melon™ Gel Purification Buffer to a column. Cap the columns and incubate for 5 min at room temperature with end-over-end rotation.
6. Remove the bottom caps from the columns, loosen the top cap and insert the spin columns into fresh 2 ml collection tubes.
7. Centrifuge for 1 min at 3,000×g to collect the purified antibody in the collection tubes.
8. Set up a new column corresponding to each sample that has been purified and repeat steps 2-7 in order to further purify the collected IgG using fresh Melon™ Gel. This portion of the procedure is repeated in order to ensure exclusive isolation of IgG from patient sera.
9. Measure the concentration of IgG using Pierce's BCA™ Protein Assay Reagent Kit.
10. Dilute each sample to 1 mg antibody/ml by adding the appropriate volume of 1× Melon™ Gel Purification Buffer. The final amount of IgG should be at least 200 μg per sample.

Labeling IgG with Fluorescent Dye

The following steps may be carried out several days prior to the rest of the experiment. However, unbound dye must be removed before storing of the labeled IgG samples (see next section of protocol). Labeled IgG can be stored for a maximum of one month at 4° C. protected from light. If labeled IgG is to be stored for longer than one month, aliquots should be protected from light in a −20° C. freezer until use. Repeated freeze/thaw cycles should be avoided.

In order to label the purified antibodies with fluorescent dyes, the antibodies must be in a buffer free of ammonium ions and primary amines. The Melon™ Gel Purification Buffer is compatible with all of the DyLight™ labeling reagents.

Complete steps 1-10 rapidly without interruption. Once the DyLight™ dyes are reconstituted, they must be used immediately.
1. Set up and label one 0.5 ml microfuge tube for each sample (four tubes total: A-DyLight™ 547, A-DyLight™ 647, B-DyLight™ 547, B-DyLight™ 647).
2. Transfer 100 μg of the appropriate purified, labeled antibody (1 mg/ml) to the corresponding tube prepared in Step 1.
3. Add 10 μl of Borate Buffer (0.67 M) to each of the 0.5 ml tubes from Step 2.
4. Tap the bottom of the DyLight™ Reagent vials against a hard surface to ensure there is no dye in the caps.
5. Reconstitute one vial of DyLight™ 547 Reagent and one vial of DyLight™ 647 Reagent by adding 20 μl of Dimethylformamide to each vial.
6. Vortex the two dye vials and briefly and centrifuge to collect the reconstituted dyes at the bottom.
7. Add 8 μl of DyLight™ 547 to each of the appropriate tubes from Step 3.
8. Add 5 μl of DyLight™ 647 to each of the appropriate tubes from Step 3.

Different volumes of the DyLight™ 547 and 647 dyes are used due to the unique affinity ratio of each dye to the antibody it is labeling.
9. Vortex the four microfuge tubes gently.
10. Briefly centrifuge the microfuge tubes to collect the samples at the bottom of the tubes.
11. Incubate the tubes for 45 min at room temperature protected from light.
12. Proceed immediately with the removal of unbound dye.

Removing Unbound Dye Using Desalting Columns

The following protocol is intended for use with the Zebra™ Desalt Spin Columns by Pierce, which come with the DyLight™ dye Labeling Kits. These columns remove free dyes, allowing accurate determination of dye-to-antibody molar ratios. The columns contain a desalting resin and molecular weight cutoff. These columns perform well in desalting small sample volumes, providing excellent protein recovery and >95% retention of small molecules and salts (<7 kD). The Protein Desalting Spin Columns, also by Pierce, are effective at removing unbound DyLight™ dye molecules and provide equal protein recovery; either type of column is appropriate for this protocol.

As long as the following steps are done quickly, desalting can be completed at room temperature. Otherwise, if access to a cold room is available, we suggest that the procedure be completed at 4° C.
1. Use two Zebra™ Desalt Spin Columns for each sample: A-DyLight™ 547, A-DyLight™ 647, B-DyLight™ 547, B-DyLight™ 647 (8 columns in total). Label each one appropriately.
2. Twist off the bottom of each column and loosen the caps before placing each one in its collection tube.
3. Centrifuge each column at 1,500×g for 1 min to remove the storage buffer. Note the side of each column where the compacted resin is slanted upwards, and be sure to place the columns in the centrifuge with this area of the column facing outward in all subsequent centrifugations.
4. Blot the bottom of the columns against a laboratory tissue to remove excess liquid.
5. Place each column into a fresh 2 ml collection tube and label two tubes for each sample: A-DyLight™ 547, A-DyLight™ 647, B-DyLight™ 547, B-DyLight™ 647 (8 column/tube assemblies in total).
6. Remove the caps from the columns.
7. Carefully apply half (˜60 μl) of each labeling reaction directly onto the center of the resin bed of its corresponding column. Each labeling reaction tube should have two corresponding desalting columns.
8. After the samples have been fully absorbed (˜2 min) apply 15 μl of ultrapure water to the resin bed of each column.
9. Centrifuge the columns for 2 min at 1,500×g to collect the desalted labeled antibodies.
10. Combine the paired desalted samples so that each sample, A-DyLight™ 547, A-DyLight™ 647, B-DyLight™ 547, B-DyLight™ 647, is consolidated into one 0.5 ml tube and has a volume of approximately 130 μl.
11. Proceed immediately with the Antibody Array Incubation.

Antibody Array Incubation with Patient IgG

The labeled IgG samples should be incubated with the microarrays in the same incubation chambers used for the Incubation with Native Antigens. Some biologically relevant antigens that were not captured by the arrays' monoclonal antibodies will be found on the walls of the incubation chamber. The greater affinity of certain IgG molecules for these antigens' epitopes limits non-specific binding of labeled IgG to the microarray slides.
1. Add 5 ml of Incubation Buffer to each of two 15 ml tubes.
2. Transfer the entire sample of IgG A-DyLight™ 547 and IgG B-DyLight™ 647 (200 μg total) to one of the 15 ml tubes prepared in step 1 and label the tube Mix 1.
3. Transfer the entire sample of IgG A-DyLight™ 647 and IgG B-DyLight™ 547 (200 μg total) to one of the 15 ml tubes prepared in step 1 and label the tube Mix 2.
3. Add the Incubation Buffer mixed with IgG A and IgG B to the appropriate incubation chamber (Mix 1 to incubation chamber 1; Mix 2 to incubation chamber 2).
4. Incubate the native protein array slide at room temperature for 45 min with gentle rocking. Every 10 min, use a pipette tip to lift one end of the slide while gently rocking the Incubation Tray.
5. Add 5 ml of Incubation Buffer to each wash chamber.
6. Transfer the slides to their respective wash chambers.
7. Incubate at room temperature for 5 min with gentle rocking.
8. Remove the buffer from the wash chambers.
9. Add 5 ml of Wash Buffer 1 to each wash chamber.
10. Incubate at room temperature for 5 min with gentle rocking.
11. Repeat Steps 7-9 using Wash Buffer 2, then Wash Buffer 3, etc., until each slide has been washed with each of the Wash Buffers 1-7.
12. Dry the slides. It is important to remove as much moisture as possible from the surface of the slides before the liquid evaporates.
   a. Using scissors or a knife, puncture a small, round hole in the bottom of the Storage Vial provided. This will facilitate the removal of excess liquid from the slides.
   b. Using gloved hands and holding the slides by their edges only, hold the slides so that the excess liquid drips toward the bottom of the array slides (the area containing the manufacturer's label) and gently touch this edge to a laboratory tissue.
   c. Position the slides, array end-up, in the empty Storage Vial. Do not touch the array surface.
   d. Cap the vial and centrifuge the slides at 1,000×g for 25 min at room temperature.
   e. Using gloved hands, uncap the vial. Remove the slides one-by-one, holding the slides by their edges and taking care not to touch the array surface.
13. Proceed immediately with Microarray Scanning.

Microarray Scanning and Quantitation

Antibody microarray slides should be scanned using a laser scanner, such as the Axon GenePix 4000B or the Perkin Elmer ScanArray 5000, according to the manufacturer's specifications. The scanner must have lasers capable of emitting excitation wavelengths between 550-557 nm and 649-652 nm. The manufacturer states that the DyLight™ 547 and 647 dyes have excitation maxima of 557 and 652 nm, respectively. The excitation maxima of the more commonly used CyDyes, 550 and 649 nm, are close to the DyLight™ maxima, and thus may be used in scanning arrays labeled with the DyLight™ dyes. ScanArray Express by Packard Biosciences or GenePix Pro by Molecular Devices is recommended for the scanning of the microarray slides. GenePix Pro is recommended for the quantitation of your results.
1. Turn on the scanner and allow the lasers to warm up. The lasers on the Perkin Elmer ScanArray 5000 require 15 min to warm-up prior to scanning your arrays.
2. Run a quick/preview scan of the entire slide in order to determine the area containing the arrayed features.
3. Create a scan protocol on the computer attached to the microarray scanner.
   a. Set the protocol to scan for the DyLight™ 547 (557 nm) and DyLight™ 647 (652 nm) flourophores. Many software packages contain preprogrammed settings for the scanning of Cy3 (550 nm) and Cy5 (649 nm) dyes. These configurations may also be used to scan the closely corresponding DyLight™ dyes.
   b. Delineate the area containing the arrayed monoclonal antibodies as the portion of the array to be scanned.
   c. If the option is available, select an area on the array that contains no arrayed features to be scanned for background intensity.
   d. Set the laser powers and PMT (photomultiplier) Gains so that the signal is high enough without being saturated. Some scanners, such as the ScanArray 5000 have the capability of running an Automatic Sensitivity Calibration that adjusts the laser power and PMT Gain based on a pre-scan of the microarray slide. We suggest the following settings for scanning with the ScanArray 5000: 550 nm: (Cy3/DyLight™ 547): PMT=62%; laser power=90%
      649 nm: (Cy5/DyLight™ 647): PMT=50%; laser power=90%
4. Insert the first slide, containing the Mix 1 autoantibodies, into the scanner. For the ScanArray 5000 the microarray surface is inserted face up.
5. Begin the scan and make sure that the signal is sufficiently high, but not saturated. It may be necessary to readjust the laser and PMT Gain settings or rerun an Automatic Sensitivity Calibration if the image is saturated or the signal is too low.
6. Save the DyLight™ 547 and 647 images as separate TIFF files. The single-file TIFF format is the most useful for quantifying data from the images. Other file formats, such as BMP and JPEG, are appropriate if the microarray images will be displayed as graphics, as in a presentation or article.
7. Scan the second microarray slide, containing the Mix 2 autoantibodies, using the same settings used to scan the first slide and save the images in the same manner.
8. Obtain the Axon Grid (GAL file) that corresponds to the lot number of the Clontech™ Ab 500 Microarray used. This information is available from the manufacturer and can be found at: http://bioinfo.clontech.com/abinfo/array-list-action.do
9. Using the GenePix Pro software, use the “Alt Y” command to open the GAL file downloaded in Step 7.
10. Open the TIFF files corresponding to your first slide with the “Alt O” command.
11. Automatically align the Axon Grid with the array features using “F8.” Use the Zoom-In feature to ensure the proper alignment of the grid with the features of your array.
12. Carry out an Automatic Analysis using the “Alt A” command.
13. Check the normalization of data by choosing the “Histogram” tab at the top of the screen. When data is perfectly normalized, the Count Ratio will be equal to 1.0. Also, view the distribution of data by choosing the “Scatter Plot” tab to ensure that there are a minimal number of outlying data points.
13. Data can be exported to Excel using the “Ctrl A” command to select all data, followed by the “Ctrl C” command to copy all data to the clipboard.
14. Data from the first slide can now be pasted into Excel with the “Ctrl V” command.
15. Repeat Steps 9-13 using the TIFF images from the second slide. When opening the TIFF files from the second slide, it is necessary to check the box next to “Replace current images” in the “Open Images” dialogue box.
16. The data acquired from the pair of microarray slides may be analyzed using appropriate biostatistical methods that normalize for variable background and dye intensities. It is useful to employ a two-stage ANOVA analysis technique in order to detect differential autoantibody reactivity by two-way clustering across multiple runs.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1-35. (canceled)

36. An assay for comparing the antibodies present in two samples of serum, blood, or plasma comprising:
a) obtaining an immobilized array of antigens wherein each antigen is attached to the surface of a solid support by a standard antibody that specifically recognizes said antigen;
b) obtaining test antibodies derived from a first sample of blood, serum or plasma, wherein said test antibodies are attached to a first detectable label;
c) obtaining control antibodies derived from a second sample of blood, serum or plasma wherein said control antibodies are attached to a second detectable label, wherein said second detectable label can be distinguished from said first detectable label after incubation with said array of immobilized antigens;
d) incubating said labeled test antibodies and said labeled control antibodies with said array of immobilized antigens;
e) after the incubation of step d), removing unbound labeled antibodies from said array of immobilized antigens; and
f) measuring the first and second detectable label associated with each immobilized antigen.

37. The assay of claim 36, wherein:
a) said first and said second detectable labels are dyes or fluorescent labels and wherein said first detectable label absorbs or fluoresces at a different wavelength than said second detectable label;
b) prior to the incubation of step d), said labeled test antibodies and said labeled control antibodies are combined into a single mixture;
c) said solid support is a glass or plastic slide; said standard antibody is a monoclonal antibody of known specificity;
d) said test antibodies are from a subject that is known to have a specific disease or condition and said control antibodies are from a subject that does not have said disease or condition.

38. The assay of claim 36, wherein:
a) said assay is used as a method for diagnosing prostate cancer in a man and wherein said test antibodies are from said man and said control antibodies are from one or more individuals that do not have prostate cancer;
b) said solid support includes one or more of the following, wherein Swiss Protein accession numbers are shown in parentheses: Cytokine-inducible serine/threonine-protein kinase (Q9H4B4); Interleukin 1, beta (P01584); Nitric oxide synthase 1 (neuronal) (P29475); Tumor suppressor p53-binding protein 2 (Q13625); Myc proto-oncogene protein (P01106); Multi PDZ domain protein MUPP1 (O75970); Diaphanous protein homolog 1 (O60610); DNA-repair protein complementing XP-A cells (P23025); citron (rho-interacting, serine/threonine kinase 21) (O14578); TIA1 cytotoxic granule-associated RNA binding protein-like 1 (Q01085); Transcription factor E2F2 (Q14209); DNA polymerase epsilon, catalytic subunit A (Q07864); Chondroitin sulfate proteoglycan 5 (neuroglycan C) (O95196); DEAD (Asp-Glu-Ala-Asp) box polypeptide 1 (Q92499); BCL2-like 1 (Q07817); Ribosomal protein S6 kinase, 70 kDa, polypeptide 1 (P23443); Calcium/calmodulin-dependent protein kinase kinase 1 alpha (Q9BQH3); Sjogren syndrome antigen B (autoantigen La) (P05455); Junction plakoglobin (P14923); Calnexin (P27824); Protein tyrosine phosphatase, receptor-type, Z polypeptide 1 (O76043); Scavenger receptor class B, member 1 (Q14016); Neurexin 1-alpha precursor (Q9ULB1); Transcription initiation factor IIB (Q00403); Neutrophil cytosol factor 2 (P19878); Microtubule-associated protein tau (P10636); Lymphocyte cytosolic protein 2 (Q13094); Far upstream element (FUSE) binding protein 1 (Q12828); Protein kinase, cAMP-dependent, regulatory, type II, beta (P31323); Protein kinase C, alpha (P17252); Mitogen-activated protein kinase 1 (P28482); Katanin p80 (WD repeat containing) subunit B1 (O60620); Tumor necrosis factor (TNF superfamily member 2) (P01375); Interferon-induced, double-stranded RNA-activated protein kinase (P19525); Gephyrin (Q9NQX3); Protein kinase C (P24723); Optineurin (Q9Y218); BCL2-associated X protein (Q07814); Phospholipase C, beta 1 (phosphoinositide-specific) (Q9NQ66); Diacylglycerol kinase, theta (P52824); CDC25C (P30307); Caspase 4, apoptosis-related cysteine protease (P49662); Cellular tumor antigen p53 (P04637); Non-POU domain containing, octamer-binding (Q15233); Doublecortin (O43602); CrmA, Serine proteinase inhibitor 2 (P07385); Amyloid beta A4 precursor protein-binding family A member 3 (O96018); and G1/S-specific cyclin D3 (P30281).

39. The method of claim 38, wherein said antigens immobilized on said solid support include one or more of the following: c-myc; MEK5; CLA-1 (CD36); FNK; p53; MUPP-1; 53BP2; and neurexen.

40. The method of claim 38, wherein said antigens immobilized on said solid support include one or more of the following, wherein Swiss Protein accession numbers are shown in parentheses: Cellular apoptosis susceptibility protein (P55060); Chomodomain helicase-DNA-binding protein 3 (Q12873); Dematin (Q08495); Branched-chain-amino-acid aminotransferase, cytosolic (P54687); Insulin-like growth factor-binding protein 3 (precursor) (P17936); L-Caldesmon (Q05682); Epoxide hydrolase 1 (P07099); Nuclear factor of activated T-cells, cytoplasmic 2 (Q13469); Transcription factor PU.1 (P17947); Mothers against decapentaplegic homolog 4 (Q13485); Non-receptor tyrosine-protein kinase TYK2 (P29597); Tumor protein p73-like (Q9UBV9); G1/S-specific cyclin-D1 (P24385); Cellular tumor antigen p53 (P04637); Pleckstrin (P08567); and Interleukin-3 (precursor) (P08700).

41. The assay of claim 36, wherein:
a) said assay is used as a method for diagnosing ovarian cancer in a woman and wherein said test antibodies are from said woman and said control antibodies are from one or more individuals that do not have ovarian cancer; and
b) said antigens immobilized on said solid support include p90 ribosomal S6 kinase (RSK).

42. The assay of claim 36, wherein:
a) said assay is used as a method for diagnosing progressive benign prostate hyperplasia (BPH) in a man and wherein said test antibodies are from said man and said control antibodies are from one or more individuals that do not have BPH; and
b) said antigens immobilized on said solid support include one or more antigens selected from the group consisting of: RB2 (Swiss Prot. accession no. Q08999); eEF-2 kinase (Swiss Prot. accession no. 000418); c-Myc (Swiss Prot. accession no P01106); GM-CSF (P04141); RSK; CRIK (Swiss Prot. accession no. 014578); Ras(Ha) (Swiss Prot. accession no. Q9BR65); Cdk2 (Swiss Prot. accession no. P24941); nNOS (Swiss Prot. acc. no. P29475); p73a (Swiss Prot. accession no. 015350); Tau-5 (Swiss Prot. accession no. P28595); and myogenin (Swiss Prot. accession no. P15173).

43. A glass or plastic plate or slide comprising monoclonal antibodies, wherein:
a) each monoclonal antibody is attached to a different site on said plate or slide; and
b) at least 90% of the immobilized antibodies are bound to antigen.

44. The glass or plastic plate or slide of claim 43, wherein there are at least 5 different antigens bound to said immobilized antibodies selected from the group consisting of: c-myc; MEK5; CLA-1 (CD36); FNK; p53; MUPP-1; 53BP2; neurexen; RSK; B2 (Swiss Prot. accession no. Q08999); eEF-2 kinase (Swiss Prot. accession no. 000418); c-Myc (Swiss Prot. accession no P01106); GM-CSF (P04141); RSK; CRIK (Swiss Prot. accession no. 014578); Ras(Ha) (Swiss Prot. accession no. Q9BR65); Cdk2 (Swiss Prot. accession no. P24941); nNOS; (Swiss Prot. accession no. P29475); p73a (Swiss Prot. accession no. 015350); Tau-5 (Swiss Prot. accession no. P28595); and myogenin (Swiss Prot. accession no. P15173).

45. The glass or plastic plate or slide of claim 43, wherein there are at least 5 different antigens bound to said immobilized antibodies selected from the group consisting of:
Cytokine-inducible serine/threonine-protein kinase (Q9H4B4); Interleukin 1, beta (P01584); Nitric oxide synthase 1 (neuronal) (P29475); Tumor suppressor p53-binding protein 2 (Q13625); Myc proto-oncogene protein (P01106); Multi PDZ domain protein MUPP1 (O75970); Diaphanous protein homolog 1 (O60610); DNA-repair protein complementing XP-A cells (P23025); citron (rho-interacting, serine/threonine kinase 21) (O14578); TIA1 cytotoxic granule-associated RNA binding protein-like 1 (Q01085); Transcription factor E2F2 (Q14209); DNA polymerase epsilon, catalytic subunit A (Q07864); Chondroitin sulfate proteoglycan 5 (neuroglycan C) (O95196); DEAD (Asp-Glu-Ala-Asp) box polypeptide 1 (Q92499); BCL2-like 1 (Q07817); Ribosomal protein S6 kinase, 70 kDa, polypeptide 1 (P23443); Calcium/calmodulin-dependent protein kinase kinase 1 alpha (Q9BQH3); Sjogren syndrome antigen B (autoantigen La) (P05455); Junction plakoglobin (P14923); Calnexin (P27824); Protein tyrosine phosphatase, receptor-type, Z polypeptide 1 (O76043); Scavenger receptor class B, member 1 (Q14016); Neurexin 1-alpha precursor (Q9ULB1); Transcription initiation factor IIB (Q00403); Neutrophil cytosol factor 2 (P19878); Microtubule-associated protein tau (P10636); Lymphocyte cytosolic protein 2 (Q13094); Far upstream element (FUSE) binding protein 1 (Q12828); Protein kinase, cAMP-dependent, regulatory, type II, beta (P31323); Protein kinase C, alpha (P17252); Mitogen-activated protein kinase 1 (P28482); Katanin p80 (WD repeat containing) subunit B1 (O60620); Tumor necrosis factor (TNF superfamily member 2) (P01375); Interferon-induced, double-stranded RNA-activated protein kinase (P19525); Gephyrin (Q9NQX3); Protein kinase C (P24723); Optineurin (Q9Y218); BCL2-associated X protein (Q07814); Phospholipase C, beta 1 (phosphoinositide-specific) (Q9NQ66); Diacylglycerol kinase, theta (P52824); CDC25C (P30307); Caspase 4, apoptosis-related cysteine protease (P49662); Cellular tumor antigen p53 (P04637); Non-POU domain containing, octamer-binding (Q15233); Doublecortin (O43602); CrmA, Serine proteinase inhibitor 2 (P07385); Amyloid beta A4 precursor protein-binding family A member 3 (O96018); and G1/S-specific cyclin D3 (P30281);
and wherein Swiss Protein accession numbers are shown in parentheses above.

46. The glass or plastic plate or slide of claim 43, wherein there are at least 5 different antigens bound to said immobilized antibodies selected from the group consisting of:
Cellular apoptosis susceptibility protein (P55060); Chomodomain helicase-DNA-binding protein 3 (Q12873); Dematin (Q08495); Branched-chain-amino-acid aminotransferase, cytosolic (P54687); Insulin-like growth factor-binding protein 3 (precursor) (P17936); L-Caldesmon (Q05682); Epoxide hydrolase 1 (P07099); Nuclear factor of activated T-cells, cytoplasmic 2 (Q13469); Transcription factor PU.1 (P17947); Mothers against decapentaplegic homolog 4 (Q13485); Non-receptor tyrosine-protein kinase TYK2 (P29597); Tumor protein p73-like (Q9UBV9); G1/S-specific cyclin-D1 (P24385); Cellular tumor antigen p53 (P04637); Pleckstrin (P08567); and Interleukin-3 (precursor) (P08700) and wherein Swiss Protein accession numbers are shown in parentheses above.

47. The glass or plastic plate or slide of claim 43, wherein there are at least 5 different antigens bound to said immobilized antibodies selected from the group consisting of: RB2 (Swiss Prot. accession no. Q08999); eEF-2 kinase (Swiss Prot. accession no. 000418); c-Myc (Swiss Prot. accession no P01106); GM-CSF (P04141); RSK; CRIK (Swiss Prot. accession no. O14578); Ras(Ha) (Swiss Prot. accession no. Q9BR65); Cdk2 (Swiss Prot. accession no. P24941); nNOS (Swiss Prot. accession no. P29475); p73a (Swiss Prot. accession no. O15350); Tau-5 (Swiss Prot. accession no. P28595); and myogenin (Swiss Prot. accession no. P15173).

48. A diagnostic assay for prostate cancer in a man, comprising:
a) obtaining a biological sample from said man;
b) determining the amount of one or more tumor associated markers (TAMs) in said biological sample, wherein said one or more TAMs is selected from the group consisting of: CLA-1 (CD36); FNK; MUPP-1; 53BP3; and neurexen; Cytokine-inducible serine/threonine-protein kinase (Q9H4B4); Interleukin 1, beta (P01584); Nitric oxide synthase 1 (neuronal) (P29475); Tumor suppressor p53-binding protein 2 (Q13625); Myc proto-oncogene protein (P01106); Multi PDZ domain protein MUPP1 (O75970); Diaphanous protein homolog 1 (O60610); DNA-repair protein complementing XP-A cells (P23025); citron (rho-interacting, serine/threonine kinase 21) (O14578); TIA1 cytotoxic granule-associated RNA binding protein-like 1 (Q0185); Transcription factor E2F2 (Q14209); DNA polymerase epsilon, catalytic subunit A (Q07864); Chondroitin sulfate proteoglycan 5 (neuroglycan C) (O95196); DEAD (Asp-Glu-Ala-Asp) box polypeptide 1 (Q92499); BCL2-like 1 (Q07817); Ribosomal protein S6 kinase, 70 kDa, polypeptide 1 (P23443); Calcium/calmodulin-dependent protein kinase kinase 1 alpha (Q9BQH3); Sjogren syndrome antigen B (autoantigen La) (P05455); Junction plakoglobin (P14923); Calnexin (P27824); Protein tyrosine phosphatase, receptor-type, Z polypeptide 1 (O76043); Scavenger receptor class B, member 1 (Q14016); Neurexin 1-alpha precursor (Q9ULB1); Transcription initiation factor IIB (Q00403); Neutrophil cytosol factor 2 (P19878); Microtubule-associated protein tau (P10636); Lymphocyte cytosolic protein 2 (Q13094); Far upstream element (FUSE) binding protein 1 (Q12828); Protein kinase, cAMP-dependent, regulatory, type II, beta (P31323); Protein kinase C, alpha (P17252); Mitogen-activated protein kinase 1 (P28482); Katanin p80 (WD repeat containing) subunit B1 (O60620); Tumor necrosis factor (TNF superfamily member 2) (P01375); Interferon-induced, double-stranded RNA-activated protein kinase (P19525); Gephyrin (Q9NQX3); Protein kinase C (P24723); Optineurin (Q9Y218); BCL2-associated X protein (Q07814); Phospholipase C, beta 1 (phosphoinositide-specific) (Q9NQ66); Diacylglycerol kinase, theta (P52824); CDC25C (P30307); Caspase 4, apoptosis-related cysteine protease (P49662); Cellular tumor antigen p53 (P04637); Non-POU domain containing, octamer-binding (Q15233); Doublecortin (O43602); CrmA, Serine proteinase inhibitor 2 (P07385); Amyloid beta A4 precursor protein-binding family A member 3 (O96018); and G1/S-specific cyclin D3 (P30281); Cellular apoptosis susceptibility protein (P55060); Chomodomain helicase-DNA-binding protein 3 (Q12873); Dematin (Q08495); Branched-chain-amino-acid aminotransferase, cytosolic (P54687); Insulin-like growth factor-binding protein 3 (precursor) (P17936); L-Caldesmon (Q05682); Epoxide hydrolase 1 (P07099); Nuclear factor of activated T-cells, cytoplasmic 2 (Q13469); Transcription factor PU.1 (P17947); Mothers against decapentaplegic homolog 4 (Q13485); Non-receptor tyrosine-protein kinase TYK2 (P29597); Tumor protein p73-like (Q9UBV9); G1/S-specific cyclin-D1 (P24385); Cellular tumor antigen p53 (P04637); Pleckstrin (P08567); and Interleukin-3 (precursor) (P08700), wherein Swiss Protein accession numbers are shown in parentheses;
c) comparing the results obtained in step b) with the amount of said one or more TAMs in a control sample derived from one or more subjects free of prostate cancer; and
d) concluding that said subject is at increased risk of having prostate cancer if the TAMs in said biological sample from said man is higher than in said control sample.

49. The diagnostic assay of claim 48, wherein said one or more TAMs is selected from the group consisting of: CLA-1 (CD36); FNK; MUPP-1; 53BP3; and neurexen.

50. The diagnostic assay of claim 48, wherein:
a) said biological sample is blood, serum or plasma;
b) said assay is a radioimmunoassay, an ELISA or a microarray assay; and
c) the amounts of at least 5 of said tumor associated markers are determined.

51. The diagnostic assay of claim 50, further comprising determining the level of prostate specific antigen (PSA) in said biological sample.

52. A diagnostic assay for ovarian cancer in a woman, comprising:
a) obtaining a test biological sample from said woman;
b) determining the amount of RSK in said test biological sample;
c) comparing the results obtained in step b) with the amount of RSK in a control sample derived from one or more subjects that do not have ovarian cancer; and
d) concluding that said woman is at increased risk of having ovarian cancer if the amount of RSK is higher in said test sample than in said control sample.

53. The diagnostic assay of claim 52, wherein:
a) said biological sample is blood, serum or plasma;
b) said assay is a radioimmunoassay, an ELISA or a microarray assay.

54. A diagnostic assay for progressive BPH in a man, comprising:
a) obtaining a biological sample from said man;
b) determining the amount of one or more tumor associated markers (TAMs) in said biological sample, wherein said one or more TAMs is selected from the group consisting of: RB2 (Swiss. Prot. accession no. Q08999); eEF-2 kinase (Swiss Prot. accession no. 000418); c-Myc (Swiss Prot. accession no P01106); GM-CSF (P04141); CRIK (Swiss Prot. accession no. O14578); Ras(Ha) (Swiss Prot. accession no. Q9BR65); Cdk2 (Swiss Prot. accession no. P24941); nNOS (Swiss Prot. accession no. P29475); p73a (Swiss Prot. accession no. O15350); Tau-5 (Swiss Prot. accession no. P28595); and myogenin (Swiss Prot. accession no. P15173);
c) comparing the results obtained in step b) with the amount of said one or more TAMs in a control sample derived from one or more subjects that do not have progressive BPH; and
d) concluding that said man is at increased risk of having progressive BPH if the TAMs in said biological sample from said man is higher than in said control sample.

55. The diagnostic assay of claim 54, wherein:
a) said biological sample is blood, serum or plasma; and
b) said diagnostic assay is a radioimmunoassay, an ELISA or a microarray assay.
